(19) United States
(12) Patent Application Publication
(10) Pub. No.: US 2014/0316213 A1
(43) Pub. Date: Oct. 23, 2014

(54) Self retaining surgical retractor

(71) Applicant: Jimmy THOMAS, (US)

(72) Inventor: Jimmy Thomas, Trivandrum (IN)

(21) Appl. No.: 14/370,760

(22) PCT Filed: Jan. 10, 2013
(86) PCT No.: PCT/IB2013/050217

§ 371 Date: Jul. 4, 2014

(30) Foreign Application Priority Data
Jan. 12, 2012 (IN) 138/CHE/2012

(51) Int. Cl. A61B 17/02

(52) U.S. Cl. CPC A61B 17/0281
USPC 600/217

(57) ABSTRACT

A surgical retractor for retracting a surgical incision, the surgical retractor includes a handle for holding the surgical retractor in position has a front end and a tail portion. The surgical retractor also has a working part which has a front end and rear end, wherein the rear end is coupled to the tail portion of the handle, thereby making an acute angle between the handle and the working. The frontend of the working part has at least one peg present, wherein the peg configured to hold the skin flap and prevents sliding of the skin flap from the peg. The handle comprises a head portion with a circular bend for hooking surgical retractor to a patient screen.

### CROSS REFERENCE TO RELATED APPLICATION

The present application is a U.S. national stage application (under 35 USC §§371) of PCT international application PCT/IB2013/050217 having an international filing date 10 Jan. 2013, which claims priority from Indian provisional application no 138/CHE/2012 filed with Indian patent Office, Chennai on 12 Jan. 2012.

### FIELD OF THE INVENTION

The present inventions generally relates to surgical retractors and in particular to abdominal self-retaining surgical retractor to retract a surgical incision, retain the incision open and retract organs and tissues inside the body through the opened incision thereby minimizing the requirement of assistants during the surgical procedure.

### BACKGROUND OF THE INVENTION

In general ‘surgical retraction’ is the act of drawing back of the body tissue. In particular the surgical retraction involves the process of making an incision, wherein the incision itself would be retracted and during surgery the internal organs and tissues are intermittently retracted through the opening created by the retracted incision.

In medical field, the surgical retractor namely the abdominal retractors are typically inserted within in the abdominal incisions during surgery to forcibly enlarge the area of incision and facilitate access within the abdomen.

Typically retractors such as manual retractor may be held by an assistant in order to maintain proper placement. Numerous retractors are available and they are typically made of metals, some examples being the O'Conner-O'Sullivan retractor, Balfour, Bookwalter, Heaney, Deaver, and Richardson retractors. During the use, the metal retractors are placed at opposing edges of a linear incision and are caused to move away from each other to effectuate incision expansion. However, the metal retractors can be a cause of concern when used in certain surgical procedures, in particular in those requiring surgical incision within the abdominal or pelvic area, due to the risk of trauma, compression or injury to the surrounding delicate soft tissue with its abundant network of vessels and nerves. The retractors composed of non-metals are disclosed in U.S. Pat. No.: 4,562,832, wherein the retractors are rigid and resistant to deformation.

In the light of the foregoing there is an ongoing need of an improved surgical retractor that performs the function of retraction efficiently, thus being especially true as it is desirable that the surgeon and his assistants have the greatest access possible to the area exposed by such incision. Furthermore, the surgical appliance free the hands of the surgeon for important duties incident to the operation other than merely holding the wound open and also shorten the surgery time, thereby allowing the surgeon to perform the same amount of work with fewer assistants.

### SUMMARY OF THE INVENTION

A surgical retractor for retracting a surgical incision, the surgical retractor includes a handle for holding the surgical retractor in position has a front end and a tail portion. The surgical retractor also has a working part which has a front end and rear end, wherein the rear end is coupled to the tail portion of the handle, thereby making an acute angle between the handle and the working. The frontend of the working part has at least one peg present, wherein the peg is configured to hold the skin flap and prevents sliding of the skin flap from the peg. The handle further comprises a head portion with a circular bend for hooking surgical retractor to a patient screen.

The present invention provides a simple self retaining surgical retractor, which could be employed in various surgical procedures with maximized exposure of the surgical sites. The present invention provides effective retraction while preventing surgical interference and injury or damage to the surrounding tissues. Furthermore the simple construction of the surgical retractor minimizes the requirement of assistants during the surgical procedures and also lessens the operating time. The surgical retractor hence finds application in various surgical procedures such as and not limited to plastic surgery, general surgery, abdominal surgery, gynaecological surgery.

### BRIEF DESCRIPTION OF DRAWING

FIG. 1 illustrates an exemplary perspective view of a surgical retractor in accordance with the embodiment of the present invention.

FIG. 2 illustrates a side view of the surgical retractor in accordance with the embodiment of the present invention.

### DETAILED DESCRIPTION OF THE INVENTION

The present invention provides a surgical retractor employed in surgical procedures. In particular the retractor is a self retaining abdominal retractor to retract a surgical incision, retain the incision open and retract organs and tissues inside the body through the opened incision thereby minimizing the requirement of assistants during the surgical procedure. The aforementioned retractor hence finds application in various surgical procedures such as and not limited to plastic surgery, general surgery, abdominal surgery, gynaecological surgery.

The surgical retractor according to the aspect of the present invention provides effective retraction while preventing surgical interference and injury or damage to the surrounding tissues.

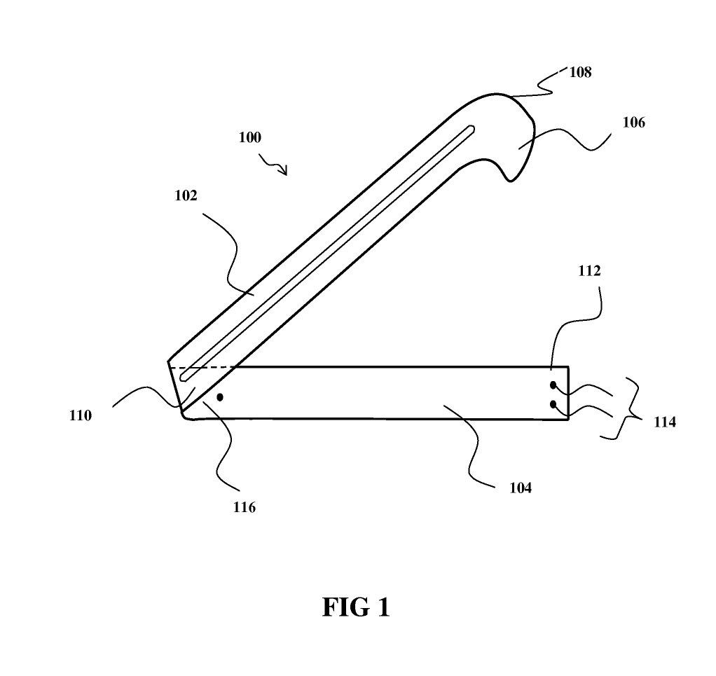

FIG. 1 illustrates an exemplary perspective view of the self retaining surgical retractor in accordance with the embodiment of the present invention. An exemplary surgical retractor (100) and its parts thereof are as illustrated in the FIG. 1. The surgical retractor (100) comprises a handle (102) for holding the surgical retractor in position. The handle has a tail portion (110) and a front end. The surgical retractor also has a working part (104) which has a front end and a rear end. The working part (104) is coupled to the tail portion of the handle (102), thereby making an acute angle between the handle (102) and the working part (104). The working part (104) may be a horizontal blade. The front end of the handle (102) of the surgical retractor (100) further comprises a head portion (106) with a circular bend (108) and the tail portion (110) coupled to the working part (104). The front end (112) of the working part (104) of the surgical retractor (100) consists of one or more pegs (114) and a rear end (116) affixed to the tail portion (110) of the handle (102). The retractor (100) is also capable of being used as hand held retractor during the surgical procedures. The pegs (114) at the front end of the working part are blunt ended.

Accordingly, the surgical retractor (100) is a single piece acute-angled surgical instrument made preferably of stainless steel, wherein the handle (102) comprises a head portion (106) with a circular bend (108) which allows the retractor (100) to be hooked on to the patient screen that renders hand free retraction or minimizes the requirement of the assistants during the operation. Adjusting the positioning of the patient screen changes the amount of retraction with ease. For instance, during the abdominal surgery the total weight of the abdominal skin flap is procured by the retractor (100) thus minimizing the requirement of the assistants during the surgical procedure. The surgical retractor according to the invention is self retaining in nature.

The handle (102) is further coupled to a working part (104) which in turn comprises a front end (112) with blunt ended pegs (114) and a rear end (116). The rear end (116) is coupled to the tail portion (110) of the handle (102) which makes the retractor (100) a single piece instrument.

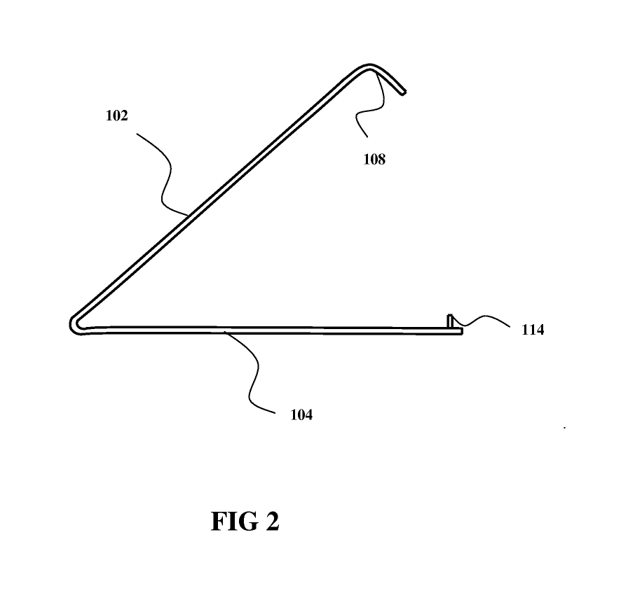

FIG. 2 illustrate the side view of the surgical retractor (100), wherein its unique acute angled shape provides effective retraction when compared to the conventional retractors.

In addition with reference to FIG. 1 the rear end (116) is coupled to the tail portion (110) of the handle (102) in such a fashion such that the angle between them provides effective retraction of the skin flap during the surgical procedures. The angle between the tail portion (110) of the handle (102) and the rear end (116) of the working part (104) also provides a clear vision towards downward the midline tunnel of the retracted skin flap during abdominal surgery, thereby enabling plication of the fascial and muscle layer with ease. Typically the plication of the fascial and muscle layer is the most strenuous during the abdominal surgery wherein the surgeon and assistants struggle to hold up the heavy abdominal flap and perform suturing simultaneously. Conventional surgical retractors employed during abdominal surgery requires additional assistants to scrub up the abdominal flap for the retraction whereas the surgical retractor (100) overcomes this drawback and allows the surgeon to perform suturing in a more relaxed and precise fashion.

Furthermore, the front end (112) of the working part (104) of the surgical retractor (100) consists of couple of blunt ended pegs (114) to hold the skin flap during the course of surgery. During the abdominal surgery the blunt ended pegs (114) prevents the slipping or sliding of the flap as the flap primarily consists of fat thereby preventing the surrounding tissues from damage.

Therefore, the present invention provides a simple self retaining surgical retractor, which could be employed in various surgical procedures with maximized exposure of the surgical sites. The present invention provides effective retraction while preventing surgical interference and injury or damage to the surrounding tissues. Furthermore the simple construction of the surgical retractor minimizes the requirement of assistants during the surgical procedures and also lessens the operating time. The surgical retractor hence finds application in various surgical procedures such as and not limited to plastic surgery, general surgery, abdominal surgery, gynaecological surgery.

I claim:

1. A surgical retractor for retracting a surgical incision, the surgical retractor comprising:
a. a handle for holding the surgical retractor in position has a front end and a tail portion;
b. a working part having a front end and rear end, wherein the rear end is coupled to the tail portion of the handle, thereby making an acute angle between the handle and the working part;
c. at least one peg present at the front end of the working part, wherein the peg is configured to hold the skin flap and prevents sliding of the skin flap from the peg.

2. The surgical retractor as claimed in claim 1, wherein the front end of the handle comprises a head portion with a circular bend for hooking the surgical retractor to a patient screen.

3. The surgical retractor as claimed in claim 1, wherein the surgical retractor is composed of stainless steel.

4. The surgical retractor as claimed in claim 1, wherein the working part is a horizontal blade.

5. The surgical retractor as claimed in claim 1, wherein the peg is blunt ended.
